SEQUENCE LISTING (10 sequences)

SEQ ID NO: 1 - DNA, 18 bp, Artificial Sequence
  sense primer for RT-PCR of IL-1beta
ctgaaagctctccacctc

SEQ ID NO: 2 - DNA, 19 bp, Artificial Sequence
  anti-sense primer for RT-PCR of IL-1beta
ggtgctgatgtaccagttg

SEQ ID NO: 3 - DNA, 14 bp, Artificial Sequence
  sense primer for RT-PCR of TNF-alpha
ccaccacgtctttg

SEQ ID NO: 4 - DNA, 18 bp, Artificial Sequence
  anti-sense primer for RT-PCR of TNF-alpha
atgggctcatacccaggg

SEQ ID NO: 5 - DNA, 19 bp, Artificial Sequence
  sense primer for RT-PCR of IDO
cactgtaccagtgcagtag

SEQ ID NO: 6 - DNA, 20 bp, Artificial Sequence
  anti-sense primer for RT-PCR of IDO
accattcacacactcgttat

SEQ ID NO: 7 - DNA, 22 bp, Artificial Sequence
  anti-sense primer for RT-PCR of iNOS
aagtcaaatcctaccaaagtga

SEQ ID NO: 8 - DNA, 21 bp, Artificial Sequence
  anti-sense primer for RT-PCR of iNOS
ccataatatggttgatgaact

SEQ ID NO: 9 - DNA, 21 bp, Artificial Sequence
  sense primer for RT-PCR of GAPDH
gaacgggaagcttgtcatcaa

SEQ ID NO: 10 - DNA, 20 bp, Artificial Sequence
  anti-sense primer for RT-PCR of GAPDH
ctaagcagttggtggtgcag

(19) United States
(12) Patent Application Publication
(10) Pub. No.: US 2007/0253961 A1
(43) Pub. Date: Nov. 1, 2007

(54) Pharmaceutical Composition Comprising the Anti-4-1Bb Antibody for Treating or Preventing Rheumatoid Arthritis

(75) Inventor: Byoung Se Kwon, Ulsan (KR)

Correspondence Address: KIRK HAHN, 14431 HOLT AVE, SANTA ANA, CA, 92705, US

(73) Assignee: ULSAN INDUSTRIAL EDUCATION FOUNDATION, Ulsan (KR)

(21) Appl. No.: 11/570,107

(22) PCT Filed: Jun. 4, 2003
(86) PCT No.: PCT/KR05/01680

§ 371 Date: Dec. 6, 2006

(30) Foreign Application Priority Data
Jun. 9, 2004 (KR) 10-2004-0042125

(51) Int. Cl. A61K 39/395

(52) U.S. Cl. 424/144.1

(57) ABSTRACT

The present invention is related to a pharmaceutical composition comprising anti-4-1BB antibody as an active ingredient in an amount effective to preventing and treating rheumatic arthritic diseases by proliferating CD1 Ic⁺CD8⁺ T cells and inducing CD4⁺ T cell suppression, together with a pharmaceutically acceptable carrier. The composition of the present invention is not toxic in general immune response and can remarkably alleviate progressive, inflammatory or auto-immune arthritis symptoms by inducing antigen-specific immune suppression. Accordingly, it can be useful in the prevention or treatment of arthritic diseases without adverse response.

### TECHNICAL FIELD

The present invention relates to pharmaceutical composition comprising a pharmaceutical composition for the treatment and prevention of rheumatoid arthritis.

### BACKGROUND ART

Rheumatoid arthritis (RA), a chronic and debilitating systemic inflammatory disease, is characterized by synovial hyperplasia and inflammatory cell recruitment, intra-articular fibrin deposition, and, in its advanced stages, cartilage and bone destruction. Although the etiology of RA remains controversial, the sequence of events in the pathogenesis of the disease as well as the end-stage effector mechanisms, appear to be well established. CD4⁺ helper T cells predominate in the initial inflammatory lesions. Macrophage-like phagocytic synovial lining cells and interdigitating synovial fibroblasts proliferate and express abundant amounts of class II major histocompatibility complex (MHC) antigens. The activated helper T cells seem to drive B cells infiltrating the synovium to produce immunoglobulins. The specificity of the majority of these locally synthesized antibodies is unknown but some are IgG rheumatoid factors that bind to other IgG molecules in the joint to form immune complexes. Finally, massive recruitment and invasion of neutrophils and macrophages, the synthesis of a battery of degradative enzymes, and the production of tumor necrosis factor-a (TNF-α), IL-1, and IL-6 erode the cartilage and other components of the rheumatoid joints (Moreland, L. W. et al. Treatment of rheumatoid arthritis with a recombinant human tumor necrosis factor receptor (p75)-Fc fusion protein. N. Engl. J. Med. 337, pp 141-147, 1997).

It is likely that these disparate disease courses converge into a common path to destruction.

The most widely used animal model for RA is collagen type II (CII)-induced arthritis (CIA), which is a Th1 cell-dependent chronic inflammation in the joints in DBA/1 mice. This model has gained acceptance since it is reproducible and well defined, and has proven useful for development of new therapies for RA, as exemplified by TNF-α neutralization treatment (Williams, R. O., Mason, L. J., Feldmann, M. & Maini, R. N. Synergy between anti-CD4 and anti-TNF in the amelioration of established collagen-induced arthritis. Proc. Natl. Acad. Sci. USA 92, pp 2762-2766, 1994). The two arms of adaptive immunity, T and B cells, play a central role in the pathogenesis of CIA but their relative importance in both priming of immune activation and joint destruction is still unclear. The major role of B cells is production of arthritogenic anti-CII antibodies, which is clearly shown by the fact that antibodies reactive with CII can bind to cartilage and induce arthritis. The role of T cells in CIA is more complex and can be divided into two main pathways that are synergistic in the development of arthritis. First, T cells provide help to B cells in the production of arthritogenic anti-CII antibodies. Second, T cells themselves play a role in joint inflammation through production of cytokines and activation of other cells.

Most of the current treatments are directed to the correction of immune aberration that supposedly drives the synovial cell proliferation and cartilage erosion. Present treatment of arthritis includes first line drugs for control of pain and inflammation classified as non-steroidal anti-inflammatory drugs (NSAIDs), e.g., aspirin, ibuprofen, naproxen, methotrexate, etc. Secondary treatments include corticosteroids, slow acting anti-rheumatic drugs (SAARDs) or disease modifying drugs (DMs), e.g., penicillinamine, cyclophosphamide, gold salts, azothipoprine, levamisole, etc. However, most potent steroidal hormone has been reported to show various adverse actions such as hyperpigmentation, amenorrhea, acne, myatonia etc till now. Recently, there have been tried to new anti-arthritic therapeutics using genetic recombination technique producing TNF receptor plays important role in inflammatory mechanism, however, there still remains a need for advanced therapeutic agent to treat and alleviate various syndrome such as inflammation, edema, abnormal angiogenesis, and bone and cartilage erosion etc till now.

4-1BB, a TNF receptor, acts on co-stimulatory receptor mainly in T lymphocyte and is induced when T cell receive antigen-specific signal. Furthermore, there have been reported that 4-1BB is expressed by other lymphoid and myeloid cell lineages such as NK (natural killer) cell, CD4⁺ CD25⁺ regulatory T cells, monocytes, and other dendritic cells (DCs). 4-1BB co-stimulates T cells to carry out effector functions such as eradication of established tumors, broadening primary CD8⁺ T cell responses, and enhancing the memory pool of antigen-specific CD8⁺ T cells. In addition, 4-1BB-mediated signals ameliorate autoimmune diseases such as systemic lupus erythematosus (SLE) and experimental autoimmune encephalitis (EAE), mainly by inhibiting CD4⁺T cell functions that drive inflammatory and T-cell-dependent antibody responses.

The importance of indoleamine 2,3-dioxygenase (IDO)-mediated immune regulation was rediscovered in the pioneering work of Munn et al. (Munn, D. H. et al. Prevention of allogeneic fetal rejection by tryptophan catabolism. Science 281, pp 1191-1193, 1998).

Therefore, the present inventors have endeavored to study the physiological action of 4-1BB and confirmed that 4-1BB-mediated suppression of RA is caused by the induction of CD11c⁺ CD8⁺ T cells in an antigen-dependent manner. Additionally we found that this novel population of induced T cells produces an abundance of IFN-γ which, in turn, induces IDO in DCs and macrophages, and antigen-specific CD4⁺ T cells are suppressed by the IDO-dependent mechanisms. Accordingly, we have accomplished the present invention by confirming that anti-4-1BB antibody is can be useful in treating or preventing arthritic disease.

### DISCLOSURE OF INVENTION

### Technical Problem

Accordingly, it is an object of the present invention to provide a pharmaceutical composition comprising anti-4-1BB antibody as an active ingredient in an amount effective to preventing and treating rheumatic arthritis by proliferating CD11c⁺CD8⁺ T cells and inducing CD4⁺ T cell suppression, together with a pharmaceutically acceptable carrier.

### Technical Solution

The pharmaceutical composition for treating arthritic diseases could contain about 0.01 to 80 w/w %, preferably 0.1 to 50 w/w % of the above anti-4-1BB antibody of present invention based on the total weight of the composition.

The anti-4-1BB antibody disclosed herein may be prepared in accordance with the procedure well known in the art. For example, hybridoma cells producing antibodies to 4-1BB (3H3) and 4-1BBL (TKS-1) can be prepared by the procedure well known in the art, for example, the preparation methods disclosed in the literatures (Shuford, W. W. et al. 4-1BB costimulatory signals preferentially induce CD8⁺ T cell proliferation and lead to amplification in vivo of cytotoxic T cell responses. J. Exp. Med. 186, pp 47-55, 1997; Futagawa, T. et al. Expression and function of 4-1BB and 4-1BB ligand on murine dendritic cells. Int. Immunol. 14, pp 275-286, 2002).

In a preferred embodiment of the present invention, to understand the role of the anti-4-1BB in the progression of collagen-induced CIA, agonistic anti-4-1BB antibody (3H3) is administrated into CIA-induced mouse and the mean clinical index of joint inflammation was observed. At the result, the development of disease was strongly inhibited by agonistic anti-4-1BB administration. All the characteristic features of CIA such as the overproduction of synovial hyperplasia, pannus formation, cartilage destruction, and bone erosion as well as various expression factors of rheumatic arthritic, i.e., chemokines such as MCP-1, MCP-2, eotaxin, MIP-1a, RANTES etc and cyokines such as IL-6, IL-15, TNF-α or IL-1β were not found and it induces to antigen-specific inhibiting reaction which completely inhibit the reproduction of IgG and IgG2b among the antibodies reacted with anti-CII.

In a preferred embodiment of the present invention, even in the progress of rheumatic arthritis, anti-4-1BB reduces disease index and inhibits the reproduction of anti-CII antibody, which enables to inhibit CIA by the 4-1BB cross-linking.

The active suppression mechanism due to anti-4-1BB antibody treatment inhibits the induction of CD4⁺ T cell and induces the increase of CD11c⁺CD8⁺ T cells within lymphonodus cell. The induced CD11c⁺CD8⁺ T cells are new CD8+ T lymphoid cells expressing CD3⁺, TCR Vβ⁺, Thy1.1⁺, CD11c and Class II antigen I-A^{q} which are different from other leukocyte surface markers such as CD11c⁻-CD8⁺, CD11c⁺ CD8⁺, CD8⁻-CD11c⁺, DCs cells. When CD11c⁺CD8⁺ T cells were isolated and adoptive transferred, they suppressed the development of CIA. Therefore, The increase CD11c⁺ CD8⁺ T cells induced by anti-4-1BB-treatment could inhibit the joint arthritic inflammation. At that time, CD11c⁺CD8⁺ T cells produce IFN-γ which induce IDO (indolamine 2,3,-dioxygenase) expression in CD11 b⁺ macrophage and CD11c⁺ dendritic cell and 1-mrthyltryptophan treatment regresses the effect of anti-4-1BB. The inhibition of CIA is caused by CD11c⁺CD8⁺ T cell proliferation and the IDO-dependent action of expressed IFN-γ suppress antigen-specific CD4⁺ T cells.

In accordance with another aspect of the present invention, there is also provided a use of anti-4-1BB antibody for the preparation of therapeutic agent for the treatment and prevention of rheumatic arthritis in a mammal including human in need thereof.

In accordance with another aspect of the present invention, there is also provided a method of treating or preventing arthritic disease in a mammal comprising administering to said mammal an effective amount of anti-4-1BB antibody, together with a pharmaceutically acceptable carrier thereof.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the composition of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing inventive composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/kg by weight/day of the inventive antibody compounds of the present invention. The dose may be administered in a single or multiple doses per day. In terms of composition, the inventive composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

### ADVANTAGEOUS EFFECTS

In accordance with another aspect of the present invention, there is also provided a use of anti-4-1BB antibody for the preparation of therapeutic agent for the treatment and prevention of rheumatic arthritis in a mammal including human in need thereof.

In accordance with another aspect of the present invention, there is also provided a method of treating or preventing arthritic disease in a mammal comprising administering to said mammal an effective amount of anti-4-1BB antibody, together with a pharmaceutically acceptable carrier thereof.

### BRIEF DESCRIPTION OF THE DRAWINGS

FIG. 1. shows clinical scores (a) and paw thickness (b) for respective test group, i.e. control IgG, anti-4-1BBL, anti-4-1BB, after immunogen challenge;

FIG. 2. shows histopathology of ankle joint after injecting control IgG(a), anti-4-1BBL(b) and anti-4-1BB(c) to CIA induced mice;

FIG. 3. presents cytokine expression for each groups i.e. control IgG, anti-4-1BBL, anti-4-1BB in ankle joints by RPA (RNase Protecting Assay);

FIG. 4. shows production of anti-CII antibodies, and serum levels in anti-CII IgG₁ treatment(a) and anti-CII IgG_{2b} treatment(b) for respective group, i.e. control IgG, anti-4-1BBL, anti-4-1BB, measured by ELISA;

FIG. 5. shows clinical scores of CIA (collagen type II-induced arthritis) for respective test group, i.e. control IgG, anti-4-1BBL, anti-4-1BB;

FIG. 6. presents levels of anti-CII IgG1(a) and anti-CII IgG2b(b) in respective test group, i.e. control IgG, anti-4-1BBL, anti-4-1BB, measured by ELISA;

FIG. 7. shows CII specific CD4⁺ T cell in respective test group, i.e. control IgG, anti-4-1BBL, anti-4-1BB, measured by [³H] marker;

FIG. 8. shows the photographs in CIA induced mice treated with CII+anti-4-1BB(a), CII+control IgG(b), and CII+anti-4-1BB in knock out mice(c), F(ab′)₂ fragment of CII-anti-4-1BB(d) and CFA+anti-4-1BB(e) after treating CII+anti-4-1BB(c) observed by FACS (Fluorescence-Activated Cell Sorter);

FIG. 9. shows cell surface marker expression of CD11c⁻CD8⁺ T cell(a), CD11c⁺ CD8⁺ T cell(b) and CD8a⁻CD11c⁺ DCs cell(c) observed by FACS;

FIG. 10. presents TCR Vβ expression spectrum of induced CD11c⁺CD8⁺ T cell;

FIG. 11. shows CD11⁺CD8⁺ T cell induction in respective test group, i.e. control IgG, anti-4-1BBL, anti-4-1BB when anti-4-1BB antibodies are injected after HSV-1 treatment;

FIG. 12. shows the increase of CD11c⁺CD8⁺ T cell(a) and CD11c⁻CD8⁺ T cell(b) at each time point in respective test group, i.e. control IgG, anti-4-1BB, after injecting CII antigen and anti-4-1BB antibody to DBA/1 mice;

FIG. 13. presents the types of induced CD11c⁺CD8⁺ T cell observed by confocal microscopy(green: anti-CD3, red: CD11c) ((a): dendritic large cell, (b): medium size dendritic cell, (c): small cell showing no dendritic morphology, (d): no dendritic morphology);

FIG. 14. shows lymph node section stained with propidium iodide after treating with CII+control IgG(a) and CII+anti-4-1BB(b);

FIG. 15. presents IFN-γ production of CD11c⁺CD8⁺ T cell induced by anti-4-1BB treatment including observation of intercellular staning(a) and culture supernatant measured by ELISA(b) for respective test group i.e. control IgG, anti-4-1BB;

FIG. 16. shows CII specific CD4⁺ T cell suppression reaction measured by [³H] marker related to the transfer of CD11c⁺CD8⁺ T cell and CD11c⁻CD8⁺ T cell for respective test group i.e. no transfer, CD11c⁻CD8⁺ T/control IgG, CD11c⁻CD8⁺ T/anti-IgG, CD11c⁺CD8⁺ T/anti-IgG;

FIG. 17. shows clinical scores after transferring CD11c⁺CD8⁺ T cell and CD11c⁻CD8⁺ T cell to new DBA/1 mice for respective test group i.e. no transfer, CD11c−CD8⁺ T/control IgG, CD11c⁻CD8⁺ T/anti-4-1BB, CD11c⁺CD8⁺ T/anti-4-1BB;

FIG. 18. presents the correlation between anti-4-1BB treatment and induction of IDO, iNOS and GAPDH by using RT-PCR and electrophoresis

(Lane 1: treating control IgG to CD11c⁻CD8⁺ T cell, Lane 2: treating anti-4-1BB to CD11c⁻CD8⁺ T cell, Lane 3: treating anti-4-1BB to CD11c⁺CD8⁺ T cell, Lane 4: treating control IgG to CD11b⁺ T cell, Lane 5: treating anti-4-1BB to CD11b⁺ T cell, Lane 6: treating control IgG to CD11c^{high} T cell, Lane 7: treating anti-4-1BB to CD11c^{high} T cell);

FIG. 19. shows the effect of IFN-γ on the induction of IDO and iNOS by using RT-PCR and electrophoresis

(Lane 1: treating control IgG to CD11b⁺ T cell, Lane 2: treating control IgG and anti-IFN-γ to CD11b⁺ T cell, Lane 3: treating anti-4-1BB to CD11b⁺ T cell, Lane 4: treating anti-4-1BB and anti-IFN-γ to CD11b⁺ T cell, Lane 5: IFN-γ treatment as positive control);

FIG. 20. shows the correlation between anti-IFN-γ treatment and CII specific CD4⁺ T cell proliferations for respective test group i.e. control IgG, control IgG/anti-IFN-γ, anti-4-1BB, anti-4-1BB/anti-IFN-γ measured by BrdU incorporation;

FIG. 21. shows clinical score after treating control IgG, anti-4-1BB, anti-4-1BB and anti-IFN-γ to CIA induced mice;

FIG. 22. presents CIA inhibitory effect of 1-methyl D,L-tryptophan(1-MT) CIA by 4-1BB including (a)average clinical score for dose-dependent of 1-MT in respective test group i.e. control IgG, anti-4-1BB/1-MT, anti-4-1BB/1-MT, anti-4-1BB/placebo and (b)average clinical score for anti-4-1BB treatment-dependent in respective test group i.e. control IgG/l-MT, anti-4-1BB/1-MT, anti-4-1BB/placebo.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following figures and examples. However, it should be understood that the present invention is not limited to these examples in any manner.

### MODE FOR THE INVENTION

The present invention is more specifically explained by the following figures and examples. However, it should be understood that the present invention is not limited to these examples in any manner.

### EXAMPLE 1

### Preparation of Antibody

Hybridoma cells producing antibodies to 4-1BB (3H3) and 4-1BBL (TKS-1)were kind gifts from Drs. Robert Mittler (Emory University, Atlanta, Ga.) and Hideo Yagita and Ko Okumura (Juntendo University, Tokyo, Japan), respectively. The antibodies were purified from ascites by protein G-column (Sigma, St. Louis, Mo.). The level of endotoxin was less than 0.05 unit by LAL assay (Cambrex, Walkersville, Md.) The binding activities of the mAbs were tested on anti-CD3 mAb-stimulated T cells or 4-1BBL transfected P815 cells. F(ab′)₂ fragments of 3H3 were purified using Sephacryl s-200HR columns (Sigma) after digestion of the antibody with pepsin. Purified rat IgG was purchased from Sigma and served as a control antibody. The following mAbs were purchased from BD PharMingen (San Diego, Calif.) for flow cytometry analysis: FITC-, PE-, PerCP-, and biotin-anti-CD8 (53-6.7); PE-anti-CD4 (GK1.5); PE- and biotin-anti-CD11c (HL3); FITC- and biotin-anti-CD11b (M1/70); PE-anti-B220 (RA3-6B2); FITC-anti-IFN-γ (XMG1.2); FITC-anti-IL10 (JESS-16E3); PE-anti-IL12 (C15.6); biotin-anti-H-2K^{g} (KH14); biotin-anti-I-A^{q} (KH 116); biotin-anti-CD80 (16-10A1); biotin-anti-CD86 (GL1); biotin-anti-CD40 (3/23); biotin-anti-CD45RA (14.8); purified anti-CD16/CD32 (2.4 G2); PE-, FITC-, and Cy-streptavidin; and a mouse Vβ TCR screening panel that contained FITC-conjugaged mAb against Vβ2, 3, 4, 5.1/5.2, 6, 7, 8.1/8.2, 8.3, 9, 10b, 11, 12, 13, 14 and 17 TCRs. FITC-anti-DEX205 (NLDG-145) was purchased from Serotec (Kidlington, Oxford, United Kingdom). DX5 was purchased from eBioscience (San Diego, Calif.)

### EXAMPLE 2

### CIA Induction and Anti 4-1BB Treatment

To understand the role of 4- 1BB in the progression of CIA, we tested whether agonistic anti-4-1BB (3H3) or a blocking anti-4-1BB ligand (anti-4-1BBL) (TKS-1) could modify CIA progression.

CIA was provoked in 6- to 7-week-old male DBA/1 mice by intradermal injection into the tail base of 100□ of bovine collagen II (CII) (Chondrex, Redmond, Wash.) emulsified in CFA. The antigen was supplemented with M. tuberculosis H37RA (2.0 mg/ml, Chondrex). The mice were examined daily for signs of joint inflammation and scored as follows: 0, normal; 1, erythema and mild swelling confined to the ankle joint; 2, erythema and mild swelling extending from the ankle joint; 3, erythema and moderate swelling extending from the ankle joint; 4, erythema and severe swelling extending from the ankle joint. The maximal arthritic score per paw was 4, and the maximal disease score per mouse was 16.

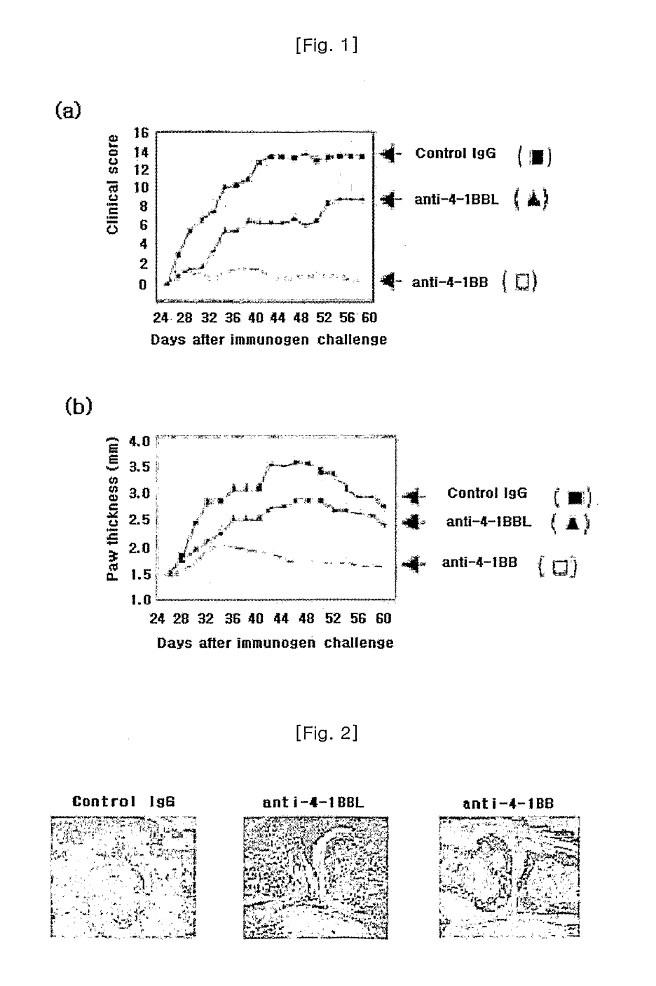

Mice treated with control IgG developed severe arthritis approximately 28 days post-immunization (PI). The severity of arthritis peaked on PI day 42 (severity=13.4±2.7; incidence=9/10; paw thickness=3.5±0.26). Mice treated with anti-4-1BBL, which blocks the interaction between 4-1BB and 4-1BBL, also developed arthritis although the disease was milder than it was in the control IgG-treated group (severity=9.6±3.2, P<0.05; incidence=8/10; paw thickness=2.7±0.25, P<0.05). Agonistic anti-4-1BB treatment strongly suppressed disease development (severity=1.6±1.6, P<0.0001; incidence=2/10; paw thickness=1.7±0.2, P<0.001) (See FIG. 1)

### EXAMPLE 3

### Histology and Immunosatining

After the immugen hind paws were fixed in 10% buffered formalin, decalcified, and embedded in paraffin. Joint sections (5-7□) were prepared and stained with hematoxylin and eosin by routine procedures. The sections were examined under a light microscope. For double fluorescence staining, purified CD11c⁺CD8⁺ T cells were incubated for 1 h on L-lysine-coated glass slides at 37° C. followed by blocking with 1□ unlabeled 2.4G2 anti-FcγR Ab (BD PharMingen). The cells were stained with FITC-anti-CD3 plus PE-anti-CD8, or FITC-anti-CD3 plus PE-anti-CD11c, or FITC-anti-CD8 plus PE-anti-CD11c. After final washing, slides were mounted in GVA mounting solution (Zymed, San Francisco, Calif.) and examined using a laser scanning confocal microscope (FV500, Olympus, Tokyo, Japan). For immunostaining of DLN, sections of DLN (8□) were washed in PBS, stained with FITC-anti-CD3 plus PE-anti-CD11c or FITC-anti-CD8 plus PE-anti-CD11c. The slides were processed as above. To minimize cross-talk between channels in the double-colored samples, a sequential scanning technique in which only one dye was excited at a time was used.

Histological examination showed that the joints of the isotype- or anti-4-1BBL-treated mice were heavily infiltrated with leukocytes, and had synovial hyperplasia, pannus formation, cartilage destruction, and bone erosion, all characteristic features of CIA. In contrast, the joints from anti-4-1BB-treated mice appeared normal and disease-free, reflecting the low severity score (See FIG. 2).

### EXAMPLE 4

### Cytokine Expression

To examine whether high level of cytokines including IL-6, IL-15, TNF-α and IL-β well-known characteristics of rheumatoid arthritis could express or not, the cytokine expression using by RPA was analyzed by isolating RNA from mouse joint tissue.

RNase Protection Assay

Total RNA was isolated from ankle tissue of each group of mice 40 days after immunization using TRIzol^{R} reagent (InVitrogen, Carlsbad, Calif.). Cytokine and chemokine mRNA levels were quantified by RNase protection assay according to the manufacture's instructions (Riboquant, BD PharMingen). Briefly, 15] of total RNA was hybridized with [³²P]UTP-labeled riboprobes (mCK-1 and mCK-5; BD PharMingen) overnight at 56° C. After hybridization, unhybridized single-stranded RNA was digested by RNase treatment. Protected RNA was then purified by phenol/chloroform extraction and ethanol precipitation. The samples were subjected to electrophoresis on a 6% polyacrylamide/7 M urea gel. The gel was dried and subjected to autoradiographic analysis.

RT-PCR

Single stranded cDNA was generated from 1□ of total RNA by using SuperscriptII (Life Technologies, Gaithersburg, Md.) and primed by oligo(dT). The cDNA was subsequently treated with 10 units of RNase-H (Life Technologies). Polymerase-chain reaction was performed in a 20□ reaction mixture with 0.5□ cDNA and 0.5 μM of each of the primers. The following primers were used:

| | |
|---|---|
| <IL-1β> | |
| Seq. I.D. 1: | |
| 5′-CTGAAAGCTCTCCACCTC-3′ | (sense primer), |
| Seq. I.D. 2: | |
| 5′-GGTGCTGATGTACCAGTTG-3′ | (anti-sense primer), |
| <TNF-α> | |
| Seq. I.D. 3: | |
| 5′-CCACCACGTCTT TG-3′ | (sense primer), |
| Seq. I.D. 4: | |
| 5′-ATGGGCTCATACCCAGGG-3′, | (anti-sense primer), |
| <GAPDH> | |
| Seq. I.D. 9: | |
| 5′-GAACGGGAAGCTTGTCATCAA-3′ | (sense primer), |
| Seq. I.D. 10: | |
| 5′CTAAGCAGTTGGTGGTGCAG-3′ | (anti-sense primer),. |

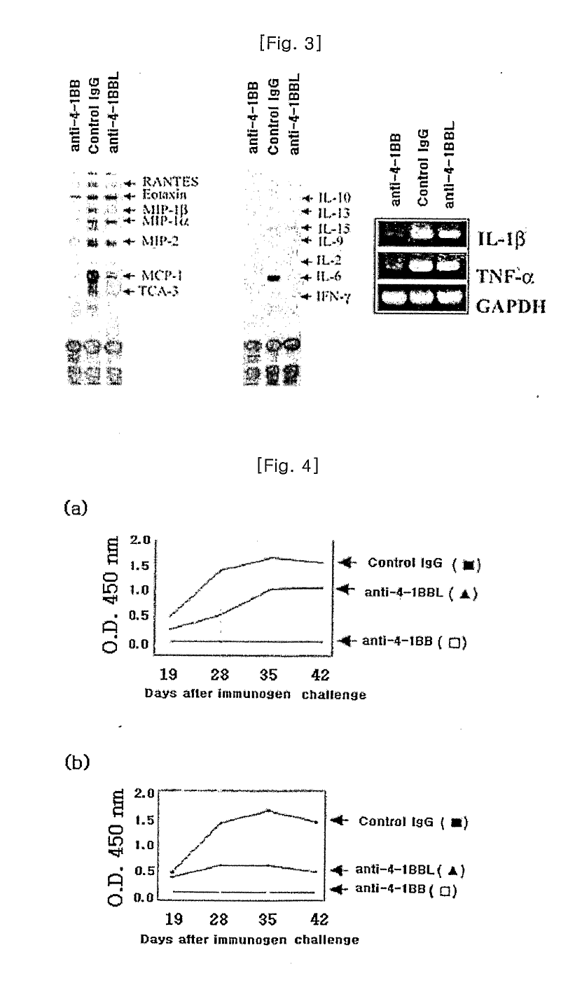

The cytokine production profiles of joint tissues also reflected disease severity: control IgG or anti-4-1BBL-treated mice had high levels of the chemokines tested, including MCP-1, MIP-2, eotaxin, MIP-1α and RANTES, whereas anti-4-1BB-treated mice had low to undetectable levels of those chemokines. Control IgG-treated mice showed a high level of IL-6, IL-15, TNF-α and IL-1 mRNA. Interestingly, anti-4-1BBL-treated mice produced high levels of IL-15, TNF-α and IL-1 but a very low level of IL-6 mRNA. In contrast, anti-4-1BB-treated mice revealed low to undetectable levels of these cytokines. High levels of IL-1β, TNF-α and IL-6 expression are well-known characteristics of rheumatoid arthritis. (See FIG. 3)

### EXAMPLE 5

### Measurement of Collagen-specific Antibody

We next examined whether anti-4-1BB treatment inhibited anti-CII antibody production.

Serum concentrations of anti-bovine CII IgG₁, IgG₂ₐ, IgG_{2b}, IgG₂, IgM, and IgE isotypes were measured by enzyme-linked immunosorbent assay (ELISA). In brief, microtiter plates were coated with bovine CII (10□/ml, Chondrex), blocked, and incubated with serially diluted test sera. Bound IgG was detected by incubation with HRP-conjugated rat anti-mouse IgG₁, IgG₂ₐ, IgG_{2b}, IgG₃, IgM, or IgE (BD PharMingen), and substrate, tetramethyl benzidine (Endogen, Rockford, Ill.). Optical densities were measured at 450 nm with an ELISA plate reader (Wallac, Turku, Finland).

Serum levels of anti-CII antibodies were measured on PI days 19, 28, 35, and 42. Anti-4-1BB treatment completely suppressed the production of anti-CII IgG₁ and IgG_{2b} (P<0.001), and anti-4-1BBL treatment somewhat decreased the production of anti-CII antibodies (P<0.05). Other isotypes of IgG, IgA, and IgE to CII were very low to undetectable (data not shown). These results again reflect disease severity.

The results of these studies in the CIA model suggest that triggering 4-1BB with anti-4-1BB induces an active suppression mechanism that is different from blocking 4-1BB/4-1BBL interactions. (See FIG. 4)

### EXAMPLE 6

### Anti-4-1BB (3H3) Ameliorates Established CIA

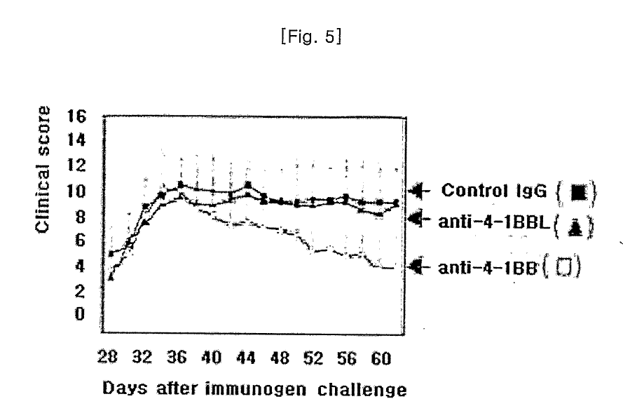

We induced CIA in DBA/1 mice, divided the mice into three groups on PI day 28 such that the mean arthritis scores of the groups were equal, and treated them with control IgG, anti-4-1BB, or anti-4-1BBL on PI days 28, 30, 32, 34, and 36 (See FIG. 5).

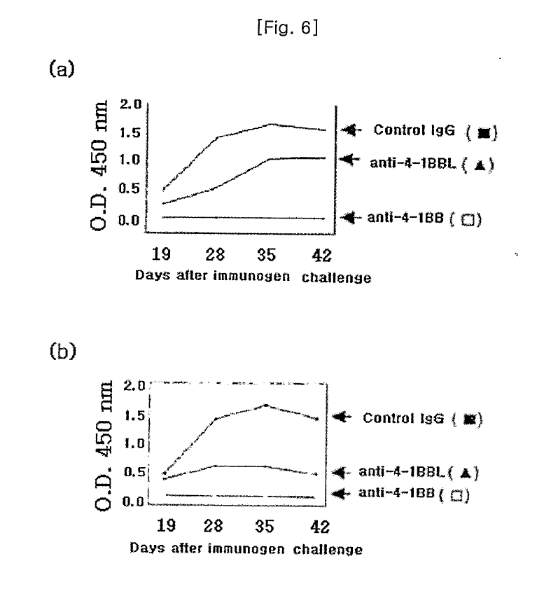

Arthritis was reversed only in the group that was treated with anti-4-1BB; this effect was significant from PI day 52 (P<0.05). Changes in CII-specific antibody levels were also examined. Anti-4-1BB treatment rapidly and almost completely cleared serum anti-CII antibodies (P<0.001). These data again demonstrate that cross-linking 4-1BB induces a suppressive mechanism against CIA (See FIG. 6).

### EXAMPLE 7

### Anti-4-1BB Suppresses CII-specific CD4⁺ T Cell Proliferation

We tested whether the active suppression induced by anti-4-1BB treatment is directed toward CD4⁺ T cells.

CD4⁺ T cells were isolated from the draining lymph nodes (DLN) of CII-immunized mice on PI day 14 and the recall response of CD4⁺ T cells to CII was examined in vitro. CD4⁺ T cells were purified by magnetic beads (Miltenyi Biotech) from DLN (axillary and inguinal lymph nodes) from control IgG, anti-4-1BB, or anti-4-1BBL mAb-treated mice on day 12 after CII immunization. Purified CD4⁺ T cells (1×10⁵) were co-cultured with mitomycin-C (Sigma)-treated (50 □/ml, 37° C., 20 min), syngeneic splenic APCs (2×10⁵) in the presence or absence of denatured CII (0.5□/ml). After incubation for 72 h at 37° C. in a 5% CO₂ atmosphere, cultures were pulsed with [³H]thymidine (1.0 μCi/well) (Amersham Pharmacia, Piscataway, N.J.) during the last 12 h. Incorporated radioactivity was counted using a scintillation counter (Wallac). Cytokine production from the above cultures was evaluated by ELISA using cytokine-specific antibody pairs from Endogen according to the manufacturer's suggestions.

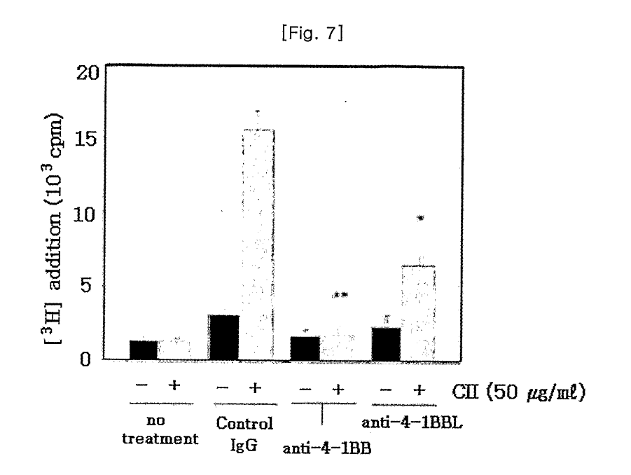

CD4⁺ T cells from the anti-4-1BB-treated mice failed to show recall responses, whereas CD4⁺ T cells from control IgG-treated mice proliferated extensively in response to CII. CD4⁺ T cells from anti-4-1BBL-treated mice in the CIA model showed a lower recall response than the control IgG-treated group (P<0.05). These data indicate that anti-4-1BB-mediated suppression is directed toward CD4⁺T cells in the CIA model (See FIG. 7).

### EXAMPLE 8

### Anti-4-1BB Treatment Induces an Expansion of the CD11c⁺ CD8⁺ T Cell Population in DLN

8-1. Flow Cytometry

To search for the mechanisms involved in 4-1BB-mediated CD4⁺ T cell suppression in the CIA model, we analyzed the subpopulations of leukocytes from DLN and spleens on PI day 12. We looked for a decrease or increase in particular populations of lymphocytes in anti-4-1BB-treated groups, compared with control IgG-treated groups. The anti-4-1BBL treatment group provided a further control and validation of the results.

We obtained red blood cell-free single cell suspensions from DLN and spleens, and subjected the lymphocytes (1×10⁶ cells in 100□) to flow cytometric analysis at 4° C. after an initial blocking step with 1□ unlabeled anti-FcγR Ab (BD PharMingen) with indicated fluorochrome-conjugated antibodies, followed by analysis by FACSCalibur (BD Biosciences). For intracellular cytokine staining, cells were stimulated with CII (50 □/ml) or CII plus-PMA (50 ng/ml; Sigma) and ionomycin (500 ng/ml; Sigma) for 18 h. GolgiPlug (BD PharMingen) was added during the last 6 h. Cells were first stained for surface markers, fixed, permeabilized, and incubated with FITC-conjugated anti-IFN-γ, anti-TGF-β, anti-IL-4, anti-IL-10, or IL-12 mAb with the Cytofix/Cytoperm Kit (BD PharMingen), according to the manufacturer's instructions.

The BrdU incorporation assay was performed according to the manufacturer's instructions (BrdU Flow Kit, BD PharMingen). Briefly, DLN cells (2×10⁵) were cultured in the presence of CII (50 □/ml). The cells were stained with PE-conjugated anti-CD4, fixed, permeabilized, treated with DNase I, and further incubated with FITC-conjugated anti-BrdU (BD PharMingen). Samples were immediately analyzed by FACS Calibur (BD Biosciences).

We examined many combinations of leukocyte surface markers by FACS and found a dramatic expansion of CD11c⁺CD8⁺cells in both DLN and spleens of anti-4-1BB-treated mice. The expression level of CD11c in this population was lower than in CD11c⁺DCs. (Although the phenotype of this population is CD11c^{low}CD8⁺, we will designate these cells as a CD11c⁺CD8⁺ cell population for simplicity.) The expansion of CD11c⁺ CD8⁺ cells was not seen in the control IgG-treated group.

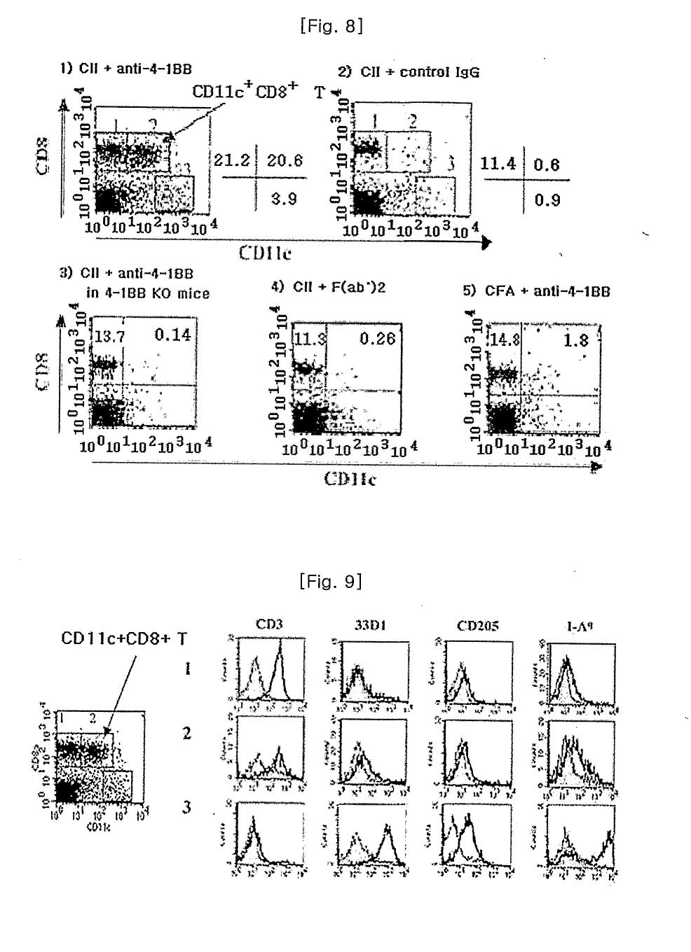

We next sought to determine the conditions that drive the expansion of CD11c⁺CD8⁺ cells. As noted above, when DBA/1 mice were immunized with CII and control IgG, there was no expansion of CD11c⁺CD8⁺ cells. When 4-1BB KO mice were immunized with CII and anti-4-1BB, there was again no expansion of this cell population, indicating that expansion required 4-1BB. When DBA/1 mice were immunized with CII and the (Fab′)₂ fragment of anti-4-1BB was injected, the CD11c⁺CD8⁺ population did not expand; expansion required 4-1BB signals generated by cross-linking with intact anti-4-1BB mAb. Finally, when DBA/1 mice were immunized with anti-4-1BB and complete Freund's adjuvant (CFA) without CII, there was no expansion. The expansion of CD11c⁺CD8⁺ cells only occurred when CII and 4-1BB signals were given together. Therefore, the expansion of CD11c⁺CD8⁺ cells is a 4-1BB-dependent and antigen-driven effect (See FIG. 8).

8-2. Phenotype of CD11c⁺ CD8⁺ Cells

To determine the phenotype of CD11c⁺CD8⁺ cells, we compared the expression of surface markers in CD11c⁻CD8⁺ T cells, CD11c⁺CD8⁺ T cells, and CD8⁻CD11c⁺ DCs. The CD11c⁺CD8⁺ cells expressed T cell markers such as CD3⁺, TCR Vβ⁺, and Thy1.2⁺. These cells differed from regular CD11c⁻CD8⁺ T cells in that they expressed the 33D1 dendritic cell marker in addition to CD11c and class II antigen I-A^{q}. They also differed from CD11c⁺CD8⁺ DCs in not expressing DC markers such as CD205, B220, and CD40, and not ingesting fluorescent dextran particles.

We concluded that this novel cell population is a subset of CD8⁺ T lymphocytes (See FIG. 9).

We also determined the TCR Vβ expression profile for the CD11c⁺CD8⁺ T cells. We immunized mice with CII to induce CIA, and then treated the mice with control anti-4-1BB. CD11c⁻CD8⁺ T cell, CD11c+CD8+ T cell and CD4⁺ T cell were isolated from DLN cells. Vβ TCR monoclonal antibody was stained with panel and analyzed by FACSCalibur (BD Biosciences, San Jose, Calif.).

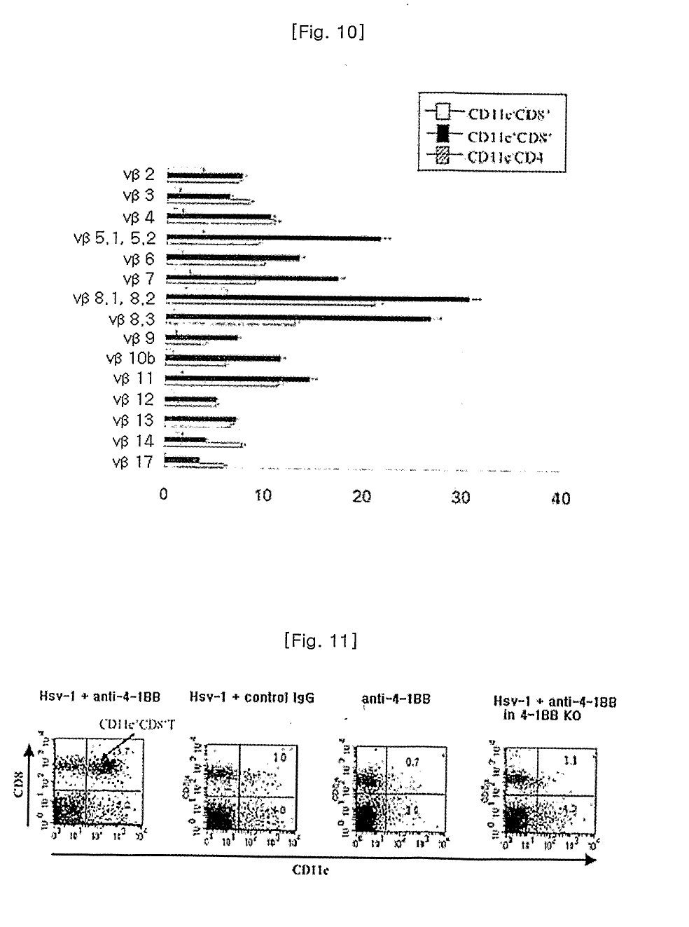

At the result, more than 30% cells show Vβ 8.1/8.2 and each Vβ 8.3, Vβ 5.1/5.2 and Vβ 7 show 28%, 22% and 17%. The VP expression spectrum in CD4⁺ T cell was similar to that in CD11c⁺CD8⁺ T cell (See FIG. 10).

8-3. Production of CD11c⁺CD8⁺ T Cells by HSV-1 Infection

We have determine whether the CD11c⁺CD8⁺ T cells are induced even when anti-4-1BB antibody is administrated after the cell has received immunogen caused by outer antigen or not.

Mice were anesthetized by i.p. injection of ketamine hydrochloride (1 mg/kg, Vetamine; Phoenix Scientific Inc., St. Joseph, Mo.) and xylazine (0.5 mg/kg, Ben Venue Laboratories, Bedford, Ohio), and infected in each hind footpad with 4×10⁵ PFU HSV-1 in 20□ of PBS. Purified anti-4-1BB (3H3, 200□) or rat IgG was injected i.p. into HSV-1-infected mice on days 0 and 2. Single cell suspensions were prepared from DLN on PI day 5. Cells were first incubated with 2.4G2 and then stained with PE-conjugated anti-mouse CD11c and Cy-conjugated anti-CD8 Abs. Stained cells were analyzed by FACSCalibur (BD Biosciences, San Jose, Calif.). A portion of the cells from HSV-1-infected mice that had been treated with control IgG or anti-4-1BB was plated on 96-well plates (5×10⁵ cells/well) and cultured in the presence or absence of 1 □/ml gB peptide for 3 days. The cells were labeled with 10 μM BrdU for 1 h and stained with PE-anti-CD4 or PE-anti-CD8 mAb, then stained intracellularly with FITC-anti-BrdU.

CD11c⁺CD8⁺ T cells were also induced by other antigens, including HSV-1 infection, when the antigens were given together with anti-4-1BB Anti-4-1BB treatment again reduced the recall response of CD4⁺ T cells to HSV-1 significantly, although the CD8⁺ T cell response was enhanced compared with the IgG-treated group (See FIG. 11).

8-4. Kinetics of Expansion and Characteristics of the CD11c⁺CD8⁺ T Cells

We have also examined the time course of the expansion of CD11c⁺CD8⁺ T cells in DBA/1 mice injected with CII.

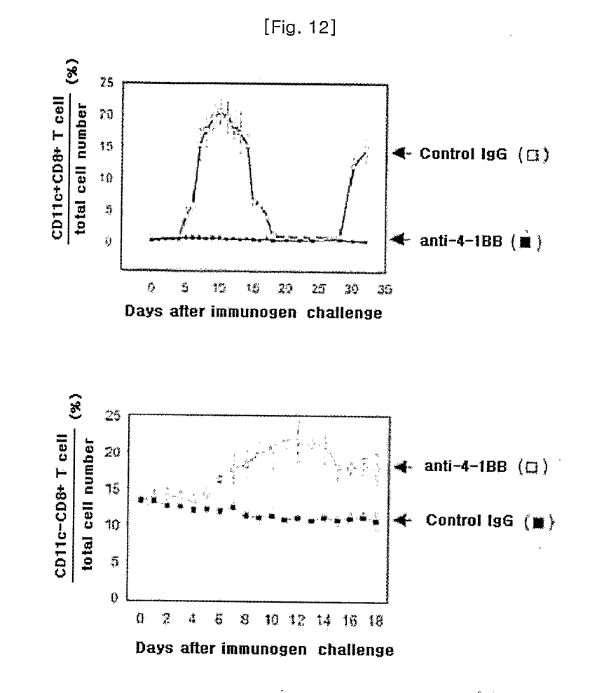

The time course of the expansion of CD11c⁺CD8⁺ T cells was determined in DBA/1 mice injected with CII and anti-4-1BB. Expansion began on PI day 5 and peaked on day 12; the cells were once more undetectable by day 18. When anti-4-1BB was administered again on PI day 24, expansion resumed after a 4-day lag. There was also an increase in the percentage of regular CD11c⁻CD8⁺ T cells (See FIG. 12).

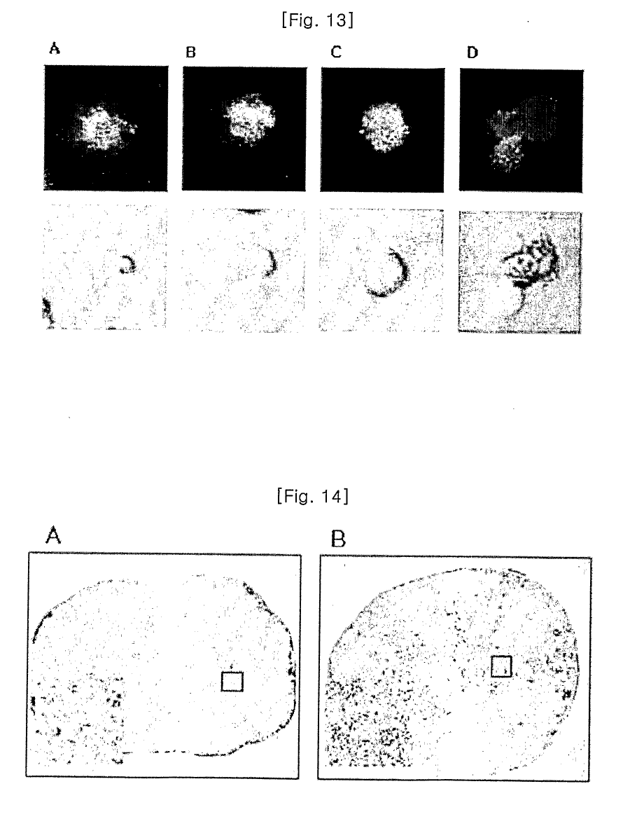

CD11c⁺CD8⁺ T cells were observed by confocal microscopy by staining with anti-CD3 and anti-CD8 or with anti-CD3 and anti-CD11c or with anti-CD8 and anti-CD11c. All three of the markers-CD3, CD8 and CD11c- were detected on the cell surface and could be merged. The expression level of CD11c was much lower than that of CD3 and CD8 (FIG. 3b1). Propidium iodide staining and FACS analysis indicated that the DNA content of the CD11c⁺CD8⁺ T cells was 2N (FIG. 3b2). Abundant CD11c+CD8+ T cells were found in lymph node sections; at their peak, these cells represented approximately 22% of the total DLN cells (See FIGS. 13 and 14).

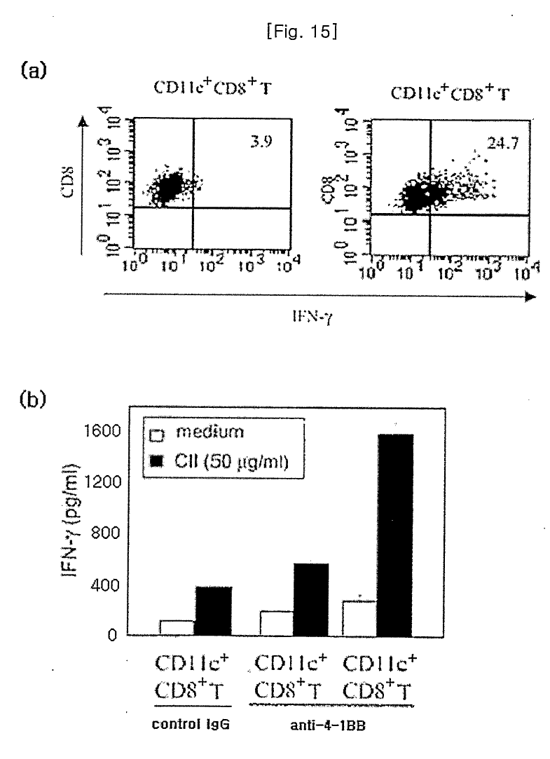

We determined cytokine production of CD11c⁺CD8⁺ T cells by intracellular staining and by ELISA, employing culture supernatant after stimulating the CD11c⁺ CD8⁺ T cells with CII. IFN-γ was the most abundant product of the CD11c⁺CD8⁺ T cells (See FIG. 15).

8-5. Treatment of CIA by Adoptive Transfer of CD11c⁺CD8⁺ T Cells.

We determined whether adoptive transfer of CD11c⁺CD8⁺ T cells suppresses CII-specific CD4⁺ T cells. CD11c⁺CD8⁺ T cells and CD11c⁻CD8⁺ T cells were prepared from DLN of CII-immunized and anti-4-1BB-treated mice, and CD11c⁻CD8⁺ T cells were also purified from CII-immunized and control IgG-treated mice. The cells were adoptively transferred into groups of new DBA/1 mice, which also received CII immunization on the same day. Seven days later, CD4⁺ T cells from the DLN were prepared from each group of mice and cultured with γ-irradiated antigen-presenting cells (APCs) in the presence or absence of CII (50 □/ml) for 72 h. Spleens were cut into small fragments and incubated in the presence of collagenase type 11 (1 mg/ml; Sigma) and DNase 1 (15 □/ml; Roche) at 37° C. for 40 min. Draining lymph nodes were incubated in the presence of 1 mg/ml collagenase and 5 mM EDTA at 37° C. for 5 min. Single cell suspensions were prepared and CD11c⁺CD8⁺ T cells were separated by MACS separation columns (Miltenyi Biotec). Because the CD11c⁺CD8⁺ T cells were negative for CD4, F4/80, CD40, and B220 cells, but the conventional CD11c⁺DCs were not (data not shown), CD11c⁺CD8⁺ T cell purification was achieved by immunomagnetically deleting the CD4⁺, F4/80, CD40⁺, and B220⁺ cell populations by incubation in a cocktail containing antibodies to these molecules. In certain experiments, DX5 specific to CD49b was included in the cocktail. Negatively selected cells (DC negative) were further incubated with anti-mouse CD11c (N418)-microbeads and separated into CD11c⁺CD8⁺ and CD11c⁻ cells. The negative cell fraction from the above step (CD11c⁻) was incubated with anti-mouse CD8 (Ly-2)-microbeads and CD11c⁻CD8⁺ cell separation was achieved by MACS columns. The purity of the selected cell population ranged between 87% and 90%. For adoptive transfer, 5×10⁶ purified cells were transferred intravenously into DBA/1 mice.

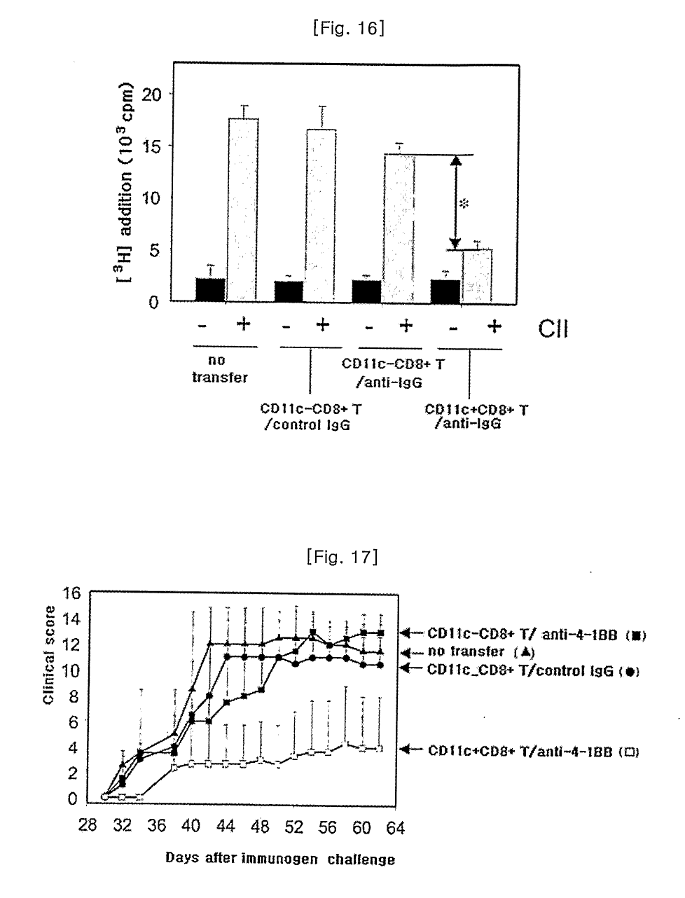

At the result, CD4⁺ T cells from the mice that received CD11c⁺CD8⁺ T cells did not demonstrate the proliferative recall response to CII, whereas the CD4⁺ T cells from the mice that received CD11c⁻CD8⁺ T cells showed normal recall responses to CII. (See FIG. 16).

Then we investigated whether the adoptive transfer of CD11c⁺CD8⁺ T cells suppressed the development of CIA. CD11c⁺CD8⁺ T cells from anti-4-1BB-treated mice or CD11c⁻CD8⁺ T cells from either anti-4-1BB-treated or control IgG-treated mice were prepared and adoptively transferred to CII-immunized DBA/1 mice on PI days 0, 10, 25, and 35. Adoptive transfer of the CD11c⁺CD8⁺ T cells ameliorated the development of CIA (P<0.001) (See FIG. 17).

### EXAMPLE 9

### Anti-IFN-γ Reverses the Anti-4-1BB-mediated Induction of IDO and iNOS and Suppression of CD4⁺ T Cells

Because CD11c⁺CD8⁺ T cells produced IFN-γ we investigated whether IFN-γ inducible effector molecules such as IDO and inducible nitric oxide synthetase (iNOS) are produced in DLN cells. IDO (Indolamine 2,3-dioxygenase) expressing cells control the immune reaction occurred by maternal T cell during pregnant period.

Specifically, CII+ control IgG and CII+anti-4-1BB were treated into mice and 14 days later, CD8⁺ T cell, CD11b⁺ monocyte, and CD11c⁺ DCs were isolated from the mice treated with control IgG. CD11c−CD8⁺ T cell, CD11c⁺CD8⁺ T cell, CD11b⁺ monocyte, and CD11c⁺ DCs were isolated from the mice treated with CII and anti-4-1BB. The RNA was extracted from the purified cells and RT-PCR was performed by using IDO, iNOS and GAPDH-specific primers. The following primers were used:

| | |
|---|---|
| <IDO> | |
| Seq. I.D. 5: | |
| 5′-CACTGTACCAGTGCAGTAG-3′ | (sense primer), |
| Seq. I.D. 6: | |
| 5′-ACCATTCACACACTCGTTAT-3′ | (anti-sense primer), |
| <iNOS> | |
| Seq. I.D. 7: | |
| 5′-AAGTCAAATCCTACC AAAGTGA-3′ | (sense primer), |
| Seq. I.D. 8: | |
| 5′-CCATAATATGGTTGATGAACT-3′ | (anti-sense primer), |
| <GAPDH> | |
| Seq. I.D. 9: | |
| 5′-GAACGGGAAGCTTGTCATCAA-3′ | (sense primer), |
| Seq. I.D. 10: | |
| 5′-CTAAGCAGTTGGTGGTGCAG-3′ | (anti-sense primer), |

Spleens from treated mice were cut into small pieces and incubated in the presence of collagenase type II (1 mg/ml; Sigma) and DNase 1 (15 □/ml; Roche Biomedicals, Mannhein, Germany) at 37° C. for 40 min. After washing, macrophage cells were purified using anti-CD 11b immunomagnetic beads and subsequent separation on MACS columns (Miltenyi Biotec, Auburn, Calif.). For isolation of DCs, cells were incubated with anti-CD90 microbeads and additional selection on MACS columns. The negatively selected cell population (CD11⁺CD8⁻) was further incubated with anti-CD11c microbeads, thus separating out the CD11c⁺ DCs. The purity of isolated macrophages and DCs was 87-90%.

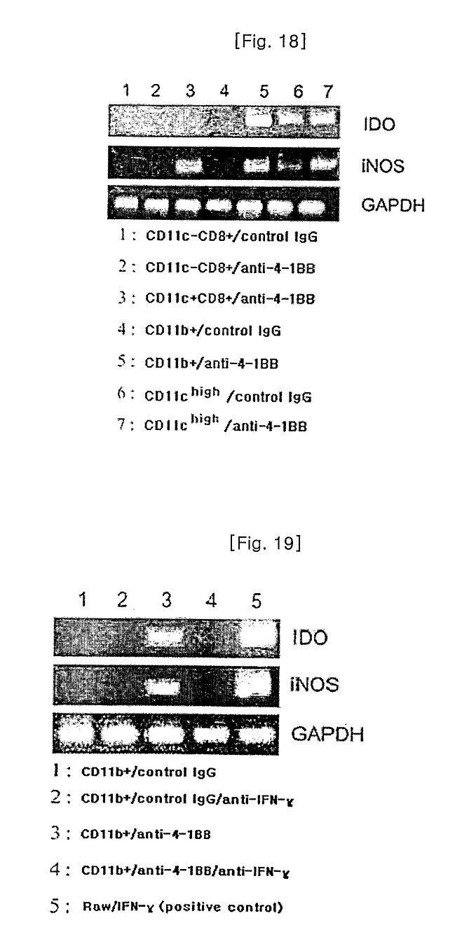

At the result, control-IgG treated mice did not produce any IDO and iNLOS mRNA and iNOS was expressed in CD11c⁺CD8⁺ T cells differently from IDO. The CD11c⁺ DC of IgG treated control mice express IDO and iNOS with low level. However, IDO and iNOS mRNA were strongly induced in CD11b⁺ monocyte as well as CD11c⁺ DC where anti-4-1BB antibody was treated (See FIG. 18).

### EXAMPLE 10

### Anti-IFN-γ Reverses the Anti-4-1BB-mediated Induction of IDO and iNOS and Suppression of CD4⁺ T Cells

To determine whether the induction of IDO and iNOS is mediated by IFN-γ CII-immunized or unimmunized mice were treated with anti-IFN-γ in combination with IgG or anti-4-1BB. We prepared macrophages and DCs from the lymph nodes of each group of mice and determined the expression of IDO and iNOS mRNA.

Specifically, we immunized DBA/1 mice with CII to induce CIA, and then treated the mice with control IgG or anti-4-1BB in combination with anti-IFN-γ CD11b⁺ monocyte was harvested from DLN cells on PI day 14 and RT-PCR for IDO, iNOS, and GAPD was performed using each RNA isolated from the CD11b+ cells treated with control IgG, both of control IgG and anti-IFN-γ anti-4-1-BB, both of anti-4-1BB and anti-IFN-γ and raw 264 cells treated with anti-IFN-γ as a positive control group.

Our results indicate that anti-IFN-γ Ab neutralized anti-4-1BB-mediated IDO and iNOS, which shows that the anti-IFN-γ Ab neutralization inhibits IDO and iNOS expression in CD11b⁺ monocyte (See FIG. 19).

To further define the involvement of IFN-γ in the suppression of CD4⁺ T cell responses to CII in anti-4-1BB-treated mice, we immunized DBA/1 mice with CII to induce CIA, and then treated the mice with control IgG or anti-4-1BB in combination with anti-IFN-γ Total DLN cells were harvested on PI day 14 and stimulated with CII in vitro.

CII-specific CD4⁺ T cell proliferation was measured by BrdU incorporation. CD4⁺

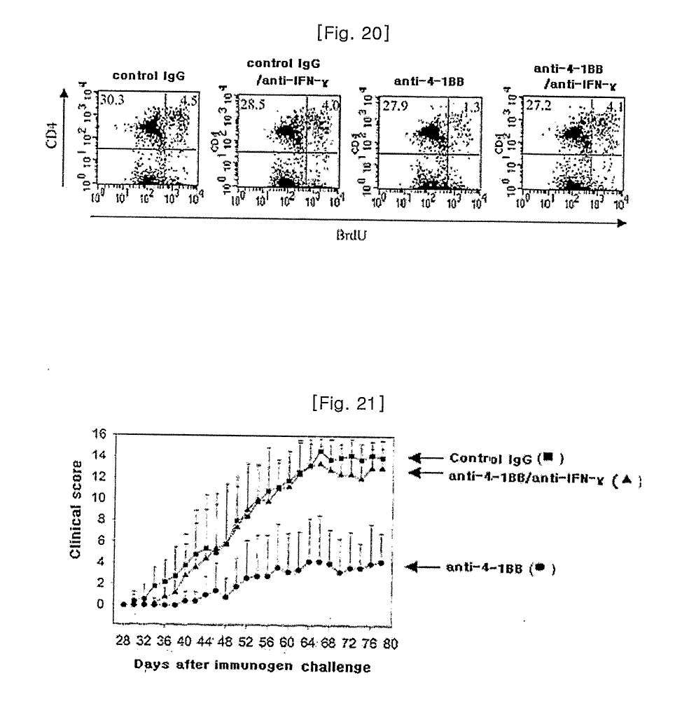

T cells from anti-4-1BB-treated mice did not proliferate in response to CII. This suppression was reversed when anti-IFN-γ was administered with anti-4-1BB (See FIG. 20).

These data suggest that the anti-arthritic effect exerted by anti-4-1BB is mediated mainly by induction of IFN-γ. The IFN-γ appears to be produced by CD11c⁺CD8⁺ T cells expanded by anti-4-1BB in combination with antigen.

To determine whether anti-IFN-γ reverses 4-1BB-mediated suppression of CIA, three groups of DBA/1 mice were immunized with CII and treated with control IgG, anti-4-1BB, or anti-4-1BB plus anti-IFN-γ. The results demonstrated that anti-IFN-γ neutralized the anti-4-1BB-mediated amelioration of CIA ( See FIG. 21).

### EXAMPLE 11

### 1-Methyltryptophan (1-MT) Completely Reverses 4-1BB-mediated Suppression of CIA

Because anti-4-1BB administration induced IDO and IDO can be a molecular effector of IFN-γ we investigated whether IDO is involved in 4-1BB-mediated suppression of CD4⁺ T cells and of CIA.

Each mouse was injected with 200□ of purified anti-4-1BB (3H3, rat IgG₁), anti-4-1BBL (TKS-1, rat IgG₁), or control rat IgG i.p. on PI days 0, 2, 4, 6, and 8. We tested the dose response of the antibodies in vivo by measuring serum IFN-γ levels for anti-4-1BB and suppression of the recall response for anti-4-1BBL. The maximal effect was observed when the antibodies were given in doses of 150 to 200□ per mouse. To treat established CIA, the mice were injected on PI days 28, 30, 32, 34, and 36. To block IFN-γ mice were injected i.p. every 4 days with 500□ of purified R4-6A2 on PI days 0, 4, 8, and 12. Rat IgG again served as a control.

To inhibit IDO activity, slow-release polymer pellets impregnated with 1-MT (10 mg/day release rate) or placebo pellets (Innovative Research of America, Sarasota, Fla.) were implanted under the dorsal skin 1 day before the immunization. In some experiments, two pellets each containing 210 mg 1-MT were implanted in each mouse, providing a dose of 20 mg/day over a 21-day period. Because of the large size of these pellets relative to the small size of the mice, additional experiments were conducted with the implantation of two smaller pellets (10 mg/day release rate) per mouse, each containing 120 mg 1-MT, for a dose of 20 mg/day for 12 days. On day 13, two more pellets were implanted, for a total dosage period of 24 days.

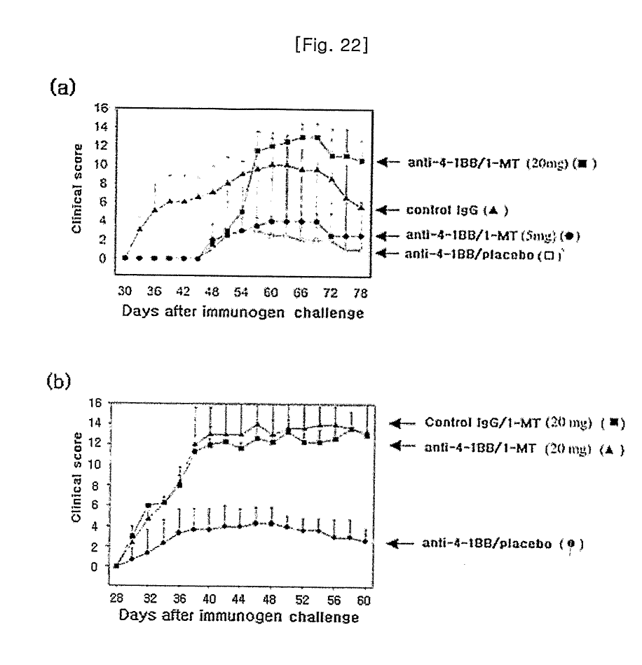

At the result, 20 mg/day dose of 1-MT completely reversed the suppressive effect of anti-4-1BB on CIA. Reversal was dose-dependent in that it was incomplete when we treated the mice with 5 mg/day. The reversal of the 3H3 effect by 1-MT was delayed; in the 1-MT-treated mice, the disease did not become evident until PI day 48, whereas control mice first showed signs of disease on PI day 30 (See FIG. 22).

In these experiments, the 1-MT reversal of the anti-4-1BB suppressive effect resulted in the appearance of CIA in these mice at the same time as in the control mice.

### EXAMPLE 12

### Toxicity Test

In order to examine the cytotoxicity of anti-4-1BB antibody, the experiment was performed as follows.

Methods

The acute toxicity on SPF Sprague-Dawley rats (Biogenomics), having its mean body weight of 108.3˜126.0, was performed using anti-4-1BB antibody. Each group consisting of 5 rats was administrated orally with 8000 mg/kg of anti-4-1BB antibody and observed for 14 days. This test was carried out in compliance with the Testing Guidelines for Safety Evaluation of Drugs (Notification No. 1999-61) issued by Korea Food and Drug Administration and the Good Laboratory Practice Regulations for Non-clinical Laboratory Studies (Notification No. 2000-63) issued by Korea Food and Drug Administration and OECD Principles of Good Laboratory Practice.

Results

There were no treatment-related effects on mortality, clinical signs, body weight changes and gross findings in any group or either gender by using 8000 mg/kg of anti-4-1BB antibody. These results suggested that the compounds prepared in the present invention were potent and safe.

Hereinafter, the formulating methods and kinds of excipients will be described, but the present invention is not limited to them. The representative preparation examples were described as follows.

Preparation of Injection

Anti-4-1BB antibody 100 mg

Sodium metabisulfite 3.0 mg

Methyl paraben 0.8 mg

Propyl paraben 0.1 mg

Distilled water for injection optimum amount

Injection preparation was prepared by dissolving active component, controlling pH to about 7.5 and then filling all the components in 2□ ample and sterilizing by conventional injection preparation method.

Preparation of Powder

Anti-4-1BB antibody 500 mg

Corn Starch 100 mg

Lactose 100 mg

Talc 10 mg

Powder preparation was prepared by mixing above components and filling sealed package.

Preparation of Tablet

Anti-4-1BB antibody 200 mg

Corn Starch 100 mg

Lactose 100 mg

Magnesium stearate optimum amount

Tablet preparation was prepared by mixing above components and entabletting.

Preparation of Capsule

Anti-4-1BB antibody 100 mg

Lactose 50 mg

Corn starch 50 mg

Talc 2 mg

Magnesium stearate optimum amount

Tablet preparation was prepared by mixing above components and filling gelatin capsule by conventional gelatin preparation method.

Preparation of Liquid

Anti-4-1BB antibody 1000 mg

Sugar 20 g

Polysaccharide 20 g

Lemon flavor 20 g

Liquid preparation was prepared by dissolving active component, and then filling all the components in 1000□ ample and sterilizing by conventional liquid preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the composition comprising inventive anti-4-1BB antibody of the present invention is not toxic in general immune response and can remarkably alleviate progressive, inflammatory or auto-immune arthritis symptoms by inducing antigen-specific immune suppression. Accordingly, it can be useful in the prevention or treatment of arthritic diseases without adverse response.

1. A pharmaceutical composition comprising anti-4-1BB antibody as an active ingredient in an amount effective for preventing and treating rheumatic arthritic diseases by proliferating CD11c⁺CD8⁺ T cells and inducing CD4⁺ T cell suppression, together with a pharmaceutically acceptable carrier.

2. The pharmaceutical composition of claim 1, wherein said composition contains about 0.01 to 50 w/w % antibody based on the total weight of the composition.

3. The pharmaceutical composition of claim 1, wherein said rheumatic arthritic disease is collagen-induced arthritis

4. A use of anti-4-1BB antibody for the preparation of therapeutic agent for the treatment and prevention of rheumatic arthritis in a mammal in need thereof.

5. The use of anti-4-1BB antibody for the preparation of therapeutic agent for the treatment and prevention of rheumatic arthritis of claim 4, wherein said mammal is a Human.
